(19) United States
(12) Patent Application Publication
(10) Pub. No.: US 2014/0301893 A1
(43) Pub. Date: Oct. 9, 2014

(54) DEVICES AND METHODS FOR STERILIZATION/DISINFECTION CONTROL OF MEDICAL DEVICES

(71) Applicant: SOTERA WIRELESS, INC., San Diego, CA (US)

(72) Inventors: David K. STROUP, El Cajon, CA (US); Arthur DEPTALA, Santee, CA (US)

(73) Assignee: SOTERA WIRELESS, INC., San Diego, CA (US)

(21) Appl. No.: 14/243,686

(22) Filed: Apr. 2, 2014

Related U.S. Application Data
(60) Provisional application No. 61/807,599, filed on Apr. 2, 2013.

(51) Int. Cl. A61L 2/10

(52) U.S. Cl. CPC A61L 2/10
USPC 422/24; 250/492.1

(57) ABSTRACT

The invention provides devices and methods for sterilization/disinfection control of medical devices. In particular, a system is provided which comprises (i) an enclosure which is configured to deliver UV light for purposes of sterilization and/or disinfection; and (ii) one or more medical devices. The enclosure and medical device(s) are configured to communicate such that the system recognizes the identity of the medical device, recognizes that sterilization/disinfection has occurred, and electronically stores data related to the sterilization/disinfection of the medical device(s) within the enclosure. This data storage can occur on the medical device(s) themselves, or on a remote computer.

### CROSS-REFERENCE TO RELATED APPLICATIONS

This application claims the benefit of U.S. Provisional Application No. 61/807,599, filed Apr. 2, 2013, which is hereby incorporated in its entirety including all tables, figures, and claims.

### BACKGROUND OF THE INVENTION

The following discussion of the background of the invention is merely provided to aid the reader in understanding the invention and is not admitted to describe or constitute prior art to the present invention.

Disinfection and sterilization methods are essential for ensuring that medical procedures and equipment do not transmit infectious pathogens to patients. Multiple studies in many countries have documented lack of compliance with established guidelines for disinfection and sterilization. Failure to comply with scientifically-based guidelines has led to numerous outbreaks. Moreover, medical equipment/devices that is not intended for single use and subsequent disposal and that cannot be cleaned and reprocessed according to the recommended guidelines for disinfection and sterilization is now often considered unsuitable for use in a medical setting.

Disinfection with ultraviolet-C (UVC) light It is known that microorganisms, such as viruses, bacteria, fungi, protozoa, algae, and so forth can be inactivated (i.e., either killed or prevented from reproducing, e.g., by molecular rearrangement of the microorganisms DNA). The wavelength of UV radiation ranges from 400 nm to 100 nm, and sterilization and disinfection methods using light of various wavelengths, including ultraviolet light of various wavelengths such as ultraviolet-C (UVC) light having a wavelength of 100 to 280 nm, ultraviolet-B (UVB) light having a wavelength 280 to 315 nm, and ultraviolet-A (UVA) light having a wavelength of 320 to 400 nm have been described. For example, UVC light has a short wavelength at which maximum bactericidal effect occurs (240-280 nm) and kills bacteria and viruses so well that it is often used to sterilize surfaces.

### SUMMARY OF THE INVENTION

It is the object of the present invention to provide devices and methods for sterilization/disinfection control of medical devices. In particular, a system is provided which comprises (i) an enclosure which is configured to deliver UV light for purposes of sterilization and/or disinfection; and (ii) one or more medical devices. The enclosure and medical device(s) are configured to communicate such that the system recognizes the identity of the medical device, recognizes that sterilization/disinfection has occurred, and electronically stores data related to the sterilization/disinfection of the medical device(s) within the enclosure. This data storage can occur on the medical device(s) themselves, or on a remote computer.

In a first aspect, the present invention provides methods for sterilization/disinfection control of medical devices. The methods comprise:
placing at least one medical device comprising a unique identifier associated therewith in an enclosure configured to deliver UV light of between 10,000-250,000 mWs/cm² within the enclosure, wherein the enclosure is operably coupled with a first processing system comprising a first transceiver and a first microprocessor to generate data to related to the sterilization/disinfection of the medical device;
sterilizing or disinfecting the medical device within the enclosure by exposing the medical device by causing the enclosure to deliver the UV light of between 10,000-250,000 mWs/cm²;
transmitting the data to related to the sterilization/disinfection of the medical device via the first transceiver to a second processing system comprising a second transceiver and a second microprocessor; and
storing the data related to the sterilization/disinfection of the medical device on an electronic storage medium associated with the second processing system, wherein the stored data is correlated to the unique identifier of the medical device.

In a related aspect, the present invention provides systems for practicing the foregoing methods. The systems comprise:
an enclosure, wherein the enclosure is configured to deliver UV light of between 10,000-250,000 mWs/cm² within the enclosure, and wherein the enclosure is operably coupled to a first processing system comprising a first transceiver and a first microprocessor, the first processing system configured to generate data to related to sterilization/disinfection cycles initiated within the enclosure and to transmit the data via the first transceiver; and
at least one medical device comprising a unique identifier associated therewith;
a second processing system comprising a second transceiver and a second microprocessor, the second processing system configured to receive the data from the first transceiver and store the data on an electronic storage medium associated with the second processing system such that the stored data is correlated to the unique identifier of the medical device.

The systems of the present invention determine the energy input for sterilization as a product of time and intensity. By way of example, a sterilization/disinfection cycle may deliver between 10,000-250,000 mWs/cm² within the enclosure, and in preferred embodiments at least 24,000 mWs/cm². Preferably the UV light is UV-C light.

The medical devices of the present invention are configured to carry a unique identifier, so that the systems of the present invention can track the sterilization/disinfection status of a particular medical device. By way of example, each device may comprise an RFID tag, RuBee tag, bar code, etc. which is interrogated by a corresponding reader. In an alternative, the unique identifier may be stored in an internal memory of the medical device, and be accessible by querying a microprocessor within the medical device which is operably coupled to the internal memory.

The unique identifier reader may be external to the enclosure, and each device may be interrogated for its unique identifier as it is placed within the enclosure. Alternatively, the reader may be within the enclosure, and the unique identifier only interrogated when the enclosure is sealed and sterilization/disinfection initiated. In the latter case, such a procedure ensures that the device is within the enclosure, and has not, for example, been scanned but not sterilized.

In certain embodiments, second processing system is carried by the medical device itself, and the unique identifier is stored in an internal memory of the medical device. In these embodiments, the first processing system may communicate with the second processing system in a wired or wireless fashion. By way of example, a medical device may be inserted into the enclosure, and the enclosure closed. Upon closure, the first processing system attempts to negotiate a connection with any medical devices that are present within the enclosure. The second first processing system on the medical device responds, and a connection is established. The second processing system can the receive data to related to the sterilization/disinfection of the medical device via the first transceiver. Preferably, such data comprises an “event stamp” such as recorded data indicating the start and/or end of the sterilization/disinfection cycle. This “event stamp” may then be stored within the internal memory of the medical device. In this manner, the data is correlated to the unique identifier of the particular medical device.

In alternative embodiments, the second processing system is provided as a remote computer system. In these embodiments, the enclosure may be operably coupled to the first processing system in a wired or wireless fashion such that the remote computer gathers the unique identifier from the medical device (e.g., by reading an RFID tag on the medical device or by connecting with a second processing system carried by the medical device which communicates with the first processing system). The remote computer system can then receive data to related to the sterilization/disinfection of the medical device via the first transceiver. Preferably, such data comprises a “event stamp” indicating the start and/or end of the sterilization/disinfection cycle. This “event stamp” may then be stored within the internal memory of the medical device. In this manner, the data is correlated to the unique identifier of the particular medical device.

As the medical device is placed into use for patient care, the device can determine from the “event stamp” the last time it has been sterilized, and if it has been used on a patient since that time. If so, it can indicate to the user by means of an alarm, light, display, etc., that it is not in a proper sterilization/disinfection state, or even going into a halt state so that it may not be used. The “event stamp” may be determined by the medical device either from an internal storage medium or by querying the remote computer, depending upon where the information is stored by the system.

The term “medical device” as used herein refers to a physical object which is used in or on a patient in order to gather medical information from the patient or render medical care. As noted above, in certain embodiments the medical device is “smart,” in that the device contains an on-board processing system and transceiver which may be exploited for communication as described above. By way of example, WO2012/092303, which is hereby incorporated by reference in its entirety, describes sensors such as ECG modules, sensors, cables, cuff-based blood pressure systems, body-worn vital sign monitors, etc., which can contain internal processors, Bluetooth transceivers and/or controller area network (CAN) bus microcontrollers which permit devices to communicate with each other without a host computer.

The term “unique identifier” as used herein refers to a label which is carried by a medical device and which unambiguously identifies the medical device to the systems of the present invention. A unique identifier may be a series of digits and/or letters as in a serial number which may be entered by hand by means of a keyboard, may be an encoded machine-readable tag, or may be an encoded identifier stored in a computer memory. Preferably, each unique identifier is not editable by an end user of the medical device, so that two medical devices may not be assigned the same identifier in error.

The term “data related to the sterilization/disinfection of the medical device” refers to any data which refers to a characteristic of a specific sterilization/disinfection cycle. Such data can include, for example, the unique identifier of a particular device within the enclosure at the time of a sterilization/disinfection cycle, the start time and/or end time of the sterilization/disinfection cycle, the intensity of light employed, the elapsed time, an expiration time after which the device should not be used without a further sterilization/disinfection cycle, whether the device has been used since the sterilization/disinfection cycle occurred, the total number of sterilization/disinfection cycles to which the medical device has been subjected, etc. This list is not meant to be limiting. Such data is “correlated” with the unique identifier for the medical device is it may be retrieved from storage and identified as applying to that particular unique identifier.

The term “electronic storage medium” refers to systems which utilize electrical power for storing and retrieving digital content. Such systems include both volatile and permanent (non-volatile) storage devices. Electronic storage media include optical media, magnetic media, flash memory, etc.

Other embodiments of the invention will be apparent from the following detailed description, exemplary embodiments, and claims.

### BRIEF DESCRIPTION OF THE DRAWINGS

FIG. 1 depicts an exemplary sterilization cabinet of the present invention.

FIG. 2 depicts insertion of medical devices into an exemplary sterilization cabinet of the present invention.

FIG. 3 depicts an interior of an exemplary sterilization cabinet of the present invention.

FIG. 4 depicts a flow chart for a sterilization method according to the present invention.

### DETAILED DESCRIPTION OF THE INVENTION

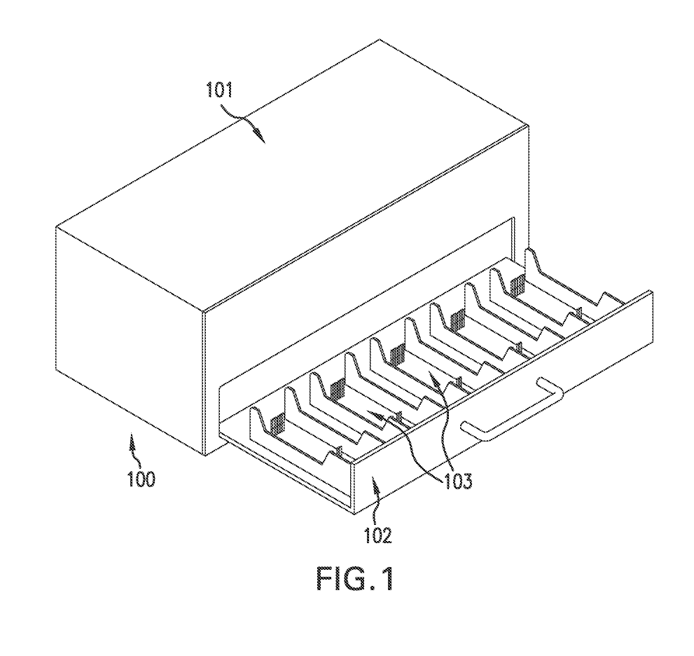

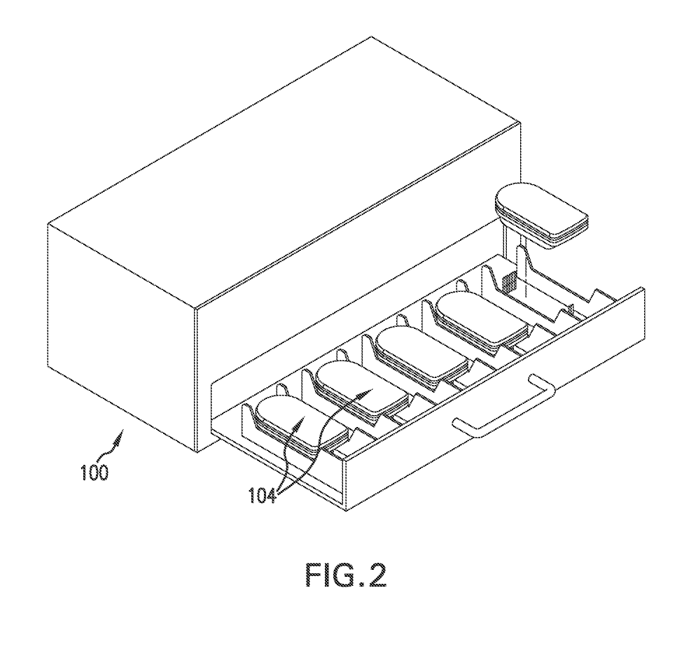

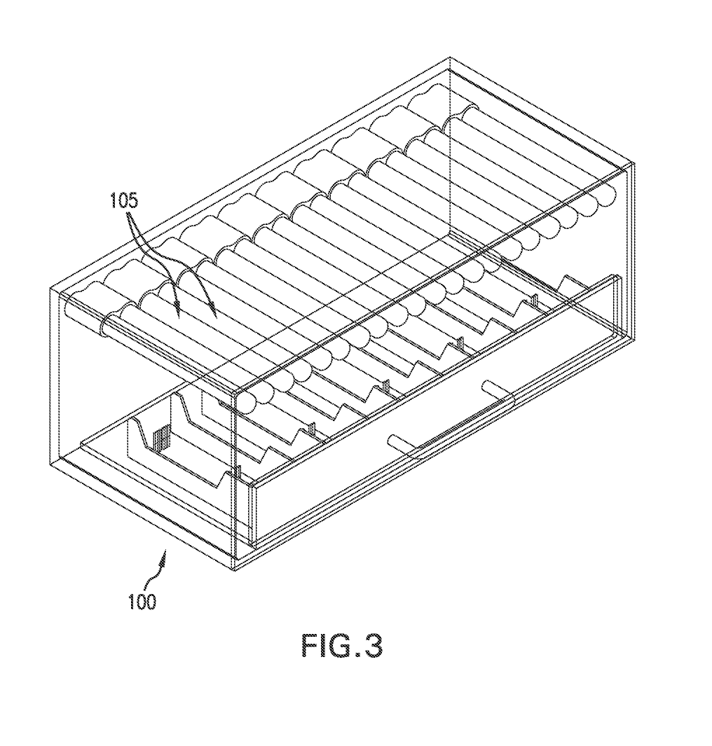

As depicted in FIG. 1, an exemplary sterilization cabinet 100 comprises an outer enclosure 101 and a drawer 102 into which medical devices are placed for sterilization. The enclosure provides shielding of the user from exposure to the UV light used for sterilization, and so is made of an opaque material which is resistant to UV light. The cabinet may be constructed similarly to those used for EPROM erasure, provided that the enclosure should include supports 103 for holding the medical devices so that all sides of the device are exposed to the UV light to ensure that no surface of the device is not sterilized. Ad depicted in FIG. 2, the medical devices placed into the enclosure are held so that they do not rest on the bottom of the enclosure, and the supports 103 can take the form of risers made from a UV transparent material. As depicted in FIG. 3, lamps 105 provide the UV source within the cabinet.

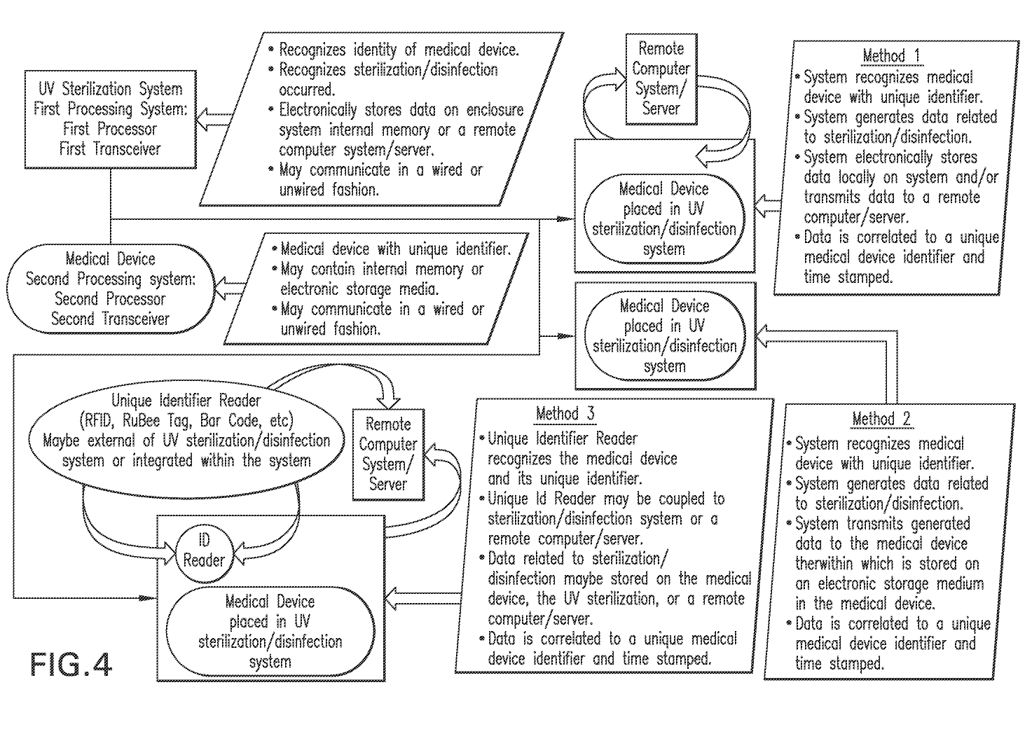

Various exemplary modes for usage of the sterilization cabinet as part of a larger sterilization system is described in FIG. 4. The sterilization cabinet is operably linked to a first processing system such that the first processing system controls, or at least recognizes, the initiation and operation of a sterilization cycle. Thus, the processing system can determine that the sterilization cabinet has been energized, and the length of time that the UV lamps are energized. This data is stored as data related to the sterilization/disinfection of any devices which are placed within the cabinet. If UV light is sufficient for sterilization (e.g., between 10,000-250,000 mWs/cm²), the first processor will record that a sterilization/disinfection cycle has completed, and can record the time at which this occurred.

In order to associate this data with particular medical devices, each device should comprise a unique identifier which may be queried. By way of example only, each device may be provided with a bar code, RFID tag, RuBee tag, memory device, etc., which comprises an encoded unique code for the device. The data related to the sterilization/disinfection is stored in association with the unique identifier of the medical devices that were the subject of the procedure. As noted in FIG. 4, this association may be accomplished in a variety of ways. For example, the encoded identifier may be read by a bar code reader, RFID tag reader, etc., operably connected to the first processing system. The reader may be a separate component, or may be included within the sterilization cabinet such that only devices within the cabinet during the sterilization/disinfection cycle are identified and associated with that cycle.

The data related to the sterilization/disinfection can provide, inter alia, data which notes a particular event. This event stamp may comprise, for example, the time of the last sterilization/disinfection cycle and/or the last use of the particular device on a patient; etc. If the device has not been sterilized/disinfected since its prior use on a patient, any attempt to use the device on another patient may be prevented by generating a warning to the user on the device itself; by refusing to power the device on; by alerting the user on a pager, cell phone, nursing station display; etc.

As noted in FIG. 4, the event data may be stored on the medical device itself. In this case, the medical device can comprise a second processing system which receives event data from the first processor coupled to the sterilization cabinet. In this case, when the medical device is subsequently used on a patient, this fact may also be noted on the device and stored in the second processing system.

Alternatively, the event data may be stored on a separate computer such as a data server comprising a second processing system. In this case, the second processing system can receive event data from the first processor coupled to the sterilization cabinet. In certain preferred embodiments, the medical device is configured to communicate with this data server when placed into use on a patient, so that proper sterilization/disinfection of the device can be assessed and patient safety maintained.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

1. A method for sterilization/disinfection control of medical devices, comprising:
placing at least one medical device comprising a unique identifier associated therewith in an enclosure configured to deliver UV light within the enclosure, wherein the enclosure is operably coupled with a first processing system comprising a first transceiver and a first microprocessor to generate data to related to the sterilization/disinfection of the medical device;
sterilizing or disinfecting the medical device within the enclosure by exposing the medical device by causing the enclosure to deliver the UV light;
transmitting the data to related to the sterilization/disinfection of the medical device via the first transceiver to a second processing system comprising a second transceiver and a second microprocessor; and
storing the data related to the sterilization/disinfection of the medical device on an electronic storage medium associated with the second processing system, wherein the stored data is correlated to the unique identifier of the medical device.

2. A method according to claim 1, wherein a sterilization/disinfection cycle delivers between 10,000-250,000 mWs/cm² of UV light within the enclosure

3. A method according to claim 1, wherein the UV light comprises UV-C light.

4. A method according to claim 1, wherein the unique identifier comprises at least one of an RFID tag, RuBee tag, or bar code, etc. which is interrogated by a corresponding reader.

5. A method according to claim 1, wherein the unique identifier comprises data stored in an internal memory of the medical device, and wherein the unique identifier is interrogated by a corresponding reader querying a microprocessor within the medical device which is operably coupled to the internal memory.

6. A method according to claim 5, wherein the reader is external to the enclosure, and the device is interrogated for its unique identifier prior to placement of the device within the enclosure.

7. A method according to claim 5, wherein the reader is within the enclosure, and the unique identifier is interrogated following placement of the device within the enclosure.

8. A method according to claim 1, wherein the second processing system is contained within the medical device.

9. A method according to claim 1, wherein the second processing system is a computer which is remote from both the medical device and the enclosure.

10. A method according to claim 1, wherein the data to related to the sterilization/disinfection of the medical device comprises data indicative of the last time the medical device has been sterilized, and/or data indicative of whether the medical device has been used on a patient since that time.

11. A system for practicing a method according to claim 1, the system comprising:
an enclosure, wherein the enclosure is configured to deliver UV light within the enclosure, and wherein the enclosure is operably coupled to a first processing system comprising a first transceiver and a first microprocessor, the first processing system configured to generate data to related to sterilization/disinfection cycles initiated within the enclosure and to transmit the data via the first transceiver; and
at least one medical device comprising a unique identifier associated therewith;
a second processing system comprising a second transceiver and a second microprocessor, the second processing system configured to receive the data from the first transceiver and store the data on an electronic storage medium associated with the second processing system such that the stored data is correlated to the unique identifier of the medical device.
